(19) United States
(12) Patent Application Publication
(10) Pub. No.: US 2007/0092471 A1
(43) Pub. Date: Apr. 26, 2007

(54) Composition for the oxidative treatment of hair or skin fixative compostion and method for permanent deformation of hair

(76) Inventors: Thorsten Cassier, Dieburg (DE); Dirk Lauscher, Seeheim-Judenheim (DE); Birgit Schreiber, Lindenfels (DE); James Kilgore, Long Beach, CA (US); Juergen Allwohn, Burgschwalbach (DE)

Correspondence Address: Striker, Striker, & Stenby, 103 East Neck Road, Huntington, NY, 11743, US

(21) Appl. No.: 10/572,790

(22) PCT Filed: Oct. 15, 2004
(86) PCT No.: PCT/EP04/11592

§ 371 Date: Mar. 21, 2006

Related U.S. Application Data
(60) Provisional application No. 60/514,413, filed on Oct. 24, 2003.

(51) Int. Cl. A61K 38/44; A61K 8/64

(52) U.S. Cl. 424/70.14; 424/94.4

(57) ABSTRACT

Cosmetic composition for the oxidative treatment of hair or skin, prepared by mixing of at least two components, in which dehydroascorbic acid or a dehydroascorbic acid salt or a dehydroascorbic acid derivative is generated from ascorbic acid, ascorbic acid derivative and ascorbic acid salt prior to application by an enzyme that catalyzes the enzymatical oxidation of said of ascorbic acid as well as a process for carrying out the oxidative treatment of keratin, particularly for the oxidative post-treatment of reduced hair in the process of permanent deformation of hair using said composition.

### BACKGROUND OF THE INVENTION

The present invention relates to a cosmetic composition for the oxidative treatment of hair or skin, prepared by mixing of at least two components prior to application, whereby dehydroascorbic acid or a dehydroascdrbic acid salt or a dehydroascorbic acid derivative is generated as well as a process for carrying out the oxidative treatment of keratin, particularly for the oxidative post-treatment of reduced hair in the process of permanent deformation of hair.

For the initial modification of keratin fibers, hair is treated with a reducing agent, which causes cleavage of the disulfide bonds of the hair protein. Reduced hair strands are brought into the desired shape and new disulfide bonds are formed. Usually, mercaptans, such as the salts or esters of mercaptocarboxylic acids, are used as reducing agents. Subsequently, the hair is rinsed with water or a suitable intermediate treatment agent. For purposes of permanent waving, the reduced hair fibers are then oxidized with a fixative. This causes new disulfide bonds to form within the hair keratin, forcing the hair to remain in the shape it had during fixation. These disulfide bonds determine the permanent durability of the deformation of the hair, especially when the hair is permanently waved or made smooth.

The most widely-used fixatives contain hydrogen peroxide, peroxide salts or bromates. When hair is treated with these compounds, a portion of the disulfide and thiol groups of the hair keratin is oxidized to higher oxidation states of sulfur, especially to cysteic acid. This means that the hair keratin is damaged irreversibly. In addition, in the case of peroxide-containing fixatives, the color pigment of the hair (melanin) is partially destroyed, resulting in lightening or bleaching of the hair.

Different fixatives based on disulfides are known, which are claimed not have these disadvantages. These alternative oxidants do, however, produce foul-smelling thiols as byproducts. Furthermore, thiol-disulfide exchange processes are equilibria, in which more free thiol groups than desired may remain on the keratin after fixation. This becomes a particular problem if, for reasons of cost, limited amounts of disulfide are used.

### SUMMARY OF THE INVENTION

It is an object of this invention to overcome several disadvantages of currently-available permanent waving formulations, especially with respect to the bleaching effect, the formation of cysteic acid and the mercaptan odor, which occur during the permanent waving of hair, and to do this without damaging the structure of the hair.

In a former invention (U.S. Pat. No. 6,506,373) it was found that this objective can be accomplished by using dehydroascorbic acid for the oxidative treatment of hair which has previously been treated reductively for permanent waving.

When producing cosmetic products for the mass market, it is desirable that the materials maintain their effectiveness when they are stored for long periods of time. Unfortunately dehydroascorbic acid is an unstable agent, which readily undergoes hydrolysis. This limits the shelf-life and adds considerably to the cost of this compound.

It is therefore desirable that dehydroascorbic acid be generated shortly before application by the oxidation of ascorbic acid, so the need for long-term storage of the complete hair treatment composition is avoided. Many efforts were made to prepare dehydroascorbic acid from ascorbic acid by using any of a number of oxidants, including Br₂, I₂, H₂O₂ and FeCl₃, and metal ion-catalyzed oxygen oxidation (e. g. Deutsch, J. C. J. Chromatogr. A, 2000, 881, 299-307). Most of these reagents are corrosive, so special facilities, equipment and operator training are required for their use. Excess reagents and corrosive byproducts (e.g. Br₂ and HBr) must be removed from the dehydroascorbic acid before its use in any preparation which is to come in contact with hair and skin. Several ascorbic acid oxidations also proceed by way of the stable ascorbate free radical and/or produce H₂O₂ as a by product, so a number of other compounds are produced in addition to dehydroascorbic acid (Deutsch, J. C. Anal. Biochem., 1998, 265, 238-245).

Producing dehydroascorbic acid in situ from ascorbic acid with H₂O₂ at room temperature according to U.S. Pat. No. 2,780,579 did not generate applicable concentrations of dehydroascorbic acid in the composition.

It has been found by the inventors that these disadvantages can be avoided by the production of dehydroascorbic acid “in situ” by enzymatic oxidation of ascorbic acid according to the equation:
2 ascorbic acid+O₂→2 dehydroascorbic acid+2 H₂O

This reaction is efficiently catalyzed by a number of enzymes, such as the ascorbate oxidases produced by most plants, as well as certain bacteria, yeasts and animals (E.C. [1.10.3.31; Lee, M. H.; Dawson, C. R. Methods Enzymol, 1979, 62,30-39).

Enzymatic oxidation of ascorbic acid has several advantages over the use of chemical oxidants, particularly the absence of corrosive reactants and reactive side-products. Furthermore, ascorbate oxidation proceeds best at slightly acidic pH values (pH 4-6), which reduce the rate of hydrolysis of dehydroascorbic acid to diketogulonic acid. The hydrolysis reaction limits the stability of dehydroascorbic acid both in solution and during storage. By oxidizing ascorbic acid immediately before performing the oxidative treatment of hair (i. e. fixative step), the need to store dehydroascorbic acid is eliminated. The enzyme, ascorbic acid and buffer solutions required for dehydroascorbic acid synthesis are nontoxic, and therefore present no hazards to workers in hair salons or to their customers.

It has now surprisingly been found that the above-mentioned disadvantages of the method of the state of the art can be avoided by proceeding according to the present invention.

It is therefore an object of the invention to provide a cosmetic composition comprising:
(a) at least one compound selected from the group consisting of ascorbic acid, ascorbic acid derivative and an ascorbic acid salt,
(b) an enzyme that catalyzes the enzymatical oxidation of said of ascorbic acid, ascorbic acid derivative or ascorbic acid salt and
(c) at least one cosmetic ingredient.

As used herein, by “cosmetic composition” is meant a solution, a creme, a paste, an ointment or a suspension containing various cosmetic ingredients typically used in formulating a composition that is applied to the skin and/or the hair. Such ingredients may include but are not limited to for example thickening agents, such as bentonite, fatty acids, starch, polyacrylic acid and its derivatives, cellulose derivatives, alginates, Vaseline, paraffin oils, wetting agents or emulsifiers from the classes of anionic, cationic, amphoteric or nonionic surface-active substances, such as fatty alcohol sulfates, fatty alcohol ether sulfates, alkylsulfonates, alkylbenzenesulfates, quaternary ammonium salts, alkylbetaines, ethoxylated alkylphenols, fatty acid alkanolamides or ethoxylated fatty esters, furthermore opacifiers, such as polyethylene glycol esters, alcohols, such as ethanol, propanol, isopropanol, polyols, such as ethylene glycol, 1,2- or 1,3-dihydroxy-propane, 1,2-, 1,3- or 1,4-dihydroxy-butane, 1,2-, 1,3-, 1,4- or 1,5-dihydroxy-pentane and glycerin, sugars, such as D-glucose, solubilizers, stabilizers, buffering substances, perfume oils, dyes as well as hair conditioning and hair caring components, such as cationic polymers, lanolin, lanolin derivatives, cholesterol, pantothenic acid and betaine.

It is preferred that said enzyme is selected from an oxygen-utilizing ascorbate oxidase. More preferred the enzyme belongs to the Enzyme Commission class [1.10.3.3]. Most preferred the enzyme is of plant origin.

Preferably the enzyme is derived from Arabidopsis, Brassica, Cucumis, Cucurbita, Myrothecium, Nicotiana, Oryza, Sinapis, Titicum species. More preferred the enzyme is derived from Cucurbita pepo medullosa (zucchini). Also very useful is highly active ascorbate oxidase purified from zucchini. Further useful is an ascorbate oxidase enzyme that has been characterized from many other plant species including cabbage (Brassica oleracea), cucumber (Cucumis sativus), pumpkin (Curcubita cv. Ebisu Nankin), tobacco (Nicotiana tabacum), mustard (Sinapis alba), rice (Oryza sativa) and wheat (Triticum aestivum). Other sources for ascorbate oxidase enzyme include fungi (Myrotecium verrucaria) and thermophilic bacteria (e.g. Acremonium sp. HI-25).

The enzyme may be present as a solution or a powder, and in either case it may be preferably stabilized by buffers, glycerol, sugars or other polyhydroxy compounds, metal chelating agents such as EDTA thiols such as thioglycerol, mercaptoethanol or dithiothreitol, polyethylene glycol, nonreactive proteins, and other common enzyme preservatives. Further stabilization of the enzyme through covalent modification is also established technology. Ascorbate oxidase, which is chemically-modified for enhanced stability, is commercially-available. Crosslinking of multimeric enzymes by reagents such as dimethyl suberimidate has also been shown to enhance stability of certain enzymes.

The enzyme may be preferably be present in immobilized form. Immobilized enzymes may be covalently attached to a solid support such as microparticles of surface-modified silica, alumina, glass, oxirane-modified polymethacrylate, carboxyalkylcellulose, aminoalkylsilica, aminoalkyl glass, aminoalkyl cellulose. Alternately, enzymes may be adsorbed on hydrophobic- or ionically-modified particle surfaces, such as carboxyalkyl- or dialkylamino-substituted cellulose. Immobilized enzymes usually display enhanced stability. An additional possibility is that the enzymes may be stabilized by covalent attachment to synthetic- or biologically-derived water soluble polymers such as polyethylene glycol (PEG), polyacrylic acid, polyvinyl alcohol, polyethyleneimine, dextran, and proteins such as gelatin or uricase. Suitable methods for covalent attachment include reaction of particle- or soluble polymer-bound aldehydes or epoxide groups with of side-chain amino groups on the enzyme, and activation of carboxyl groups either on a solid or soluble support, or on the enzyme (aspartic and glutamic side-chains) to react with enzyme sidechain amino groups or support-linked amino groups, respectively. Polyethylene glycol chains can be attached to sidechain amino groups by alkylation with PEG-derived alkylsulfonate esters and by reductive amination with PEG-derived aldehydes, among other methods.

Said enzyme is contained in the cosmetic composition in a concentration of from about 1 to about 10,000 ppm, preferably from about 10 to about 1000 ppm, whereas this is the concentration of the enzyme protein, e. g. ascorbate oxidase protein, in the ready-to-use composition immediately after mixing of all components of said composition.

The cosmetic ingredient contained in the ready-to-use cosmetic composition is selected from the group consisting of swelling and penetration materials, such as urea, 2-pyrrolidone, 1-methyl-2-pyrrolidone and dipropylene glycol monomethyl ether, as well as peroxide stabilizers, such as aromatic sulfonic acids, hydrochloric acid, sulfuric acid, phosphoric acid, pyro- or polyphosphoric acids, acidic salts, strong acids, ascorbic acid, oxalic acid, malonic acid, benzoic acid, salicylic acid, citric acid, tannic acids, paraformaldehyde, 4-acetamido-phenol, phenol, thymol or alpha-bisabolol, thickening agents, such as bentonite, kaolin, fatty acids, starch, guar gum, high molecular weight fatty alcohols, polyacrylic acid and its derivatives, cellulose derivatives, alginates, Vaseline, paraffin oils, wetting agents or emulsifiers from the classes of anionic, cationic, amphoteric, zwitterionic or nonionic surface-active substances, such as fatty alcohol sulfates, fatty alcohol ether sulfates, alkylsulfonates, alkylbenzenesulfates, quaternary ammonium salts, alkylbetaines, ethoxylated alkylphenols, fatty acid alkanolamides or ethoxylated fatty esters, furthermore opacifiers, such as polyethylene glycol esters, alcohols, such as ethanol, propanol, isopropanol, polyols, such as ethylene glycol, 1,2- or 1,3-dihydroxy-propane, 1,2-, 1,3- or 1,4-dihydroxy-butane, 1,2-, 1,3-, 1,4- or 1,5-dihydroxy-pentane and glycerin, sugars, such as D-glucose, solubilizers, stabilizers, buffering substances, perfume oils, dyes as well as hair conditioning and hair care components, such as cationic polymers e. g.
CTFA: POLYQUATERNIUM-1, CTFA: POLYQUATERNIUM-4,
CTFA: POLYQUATERNIUM-5, CTFA: POLYQUATERNIUM-6,
CTFA: POLYQUATERNIUM-7, CTFA: POLYQUATERNIUM-10,
CTFA: POLYQUATERNIUM-11, CTFA: POLYQUATERNIUM-16,
CTFA: POLYQUATERNIUM-22, CTFA: POLYQUATERNIUM-32,
CTFA: POLYQUATERNIUM-35, CTFA: POLYQUATERNIUM-36,
CTFA: POLYQUATERNIUM-37, CTFA: POLYQUATERNIUM-39,
CTFA: POLYQUATERNIUM-44, CTFA: POLYQUATERNIUM-45,
CTFA: POLYQUATERNIUM-46, CTFA: POLYQUATERNIUM-47,
   silicone polymers e. g. CTFA: POLYSILICONE-3, CTFA: POLYSILICONE-4, CTFA: POLYSILICONE-5, CTFA: POLYSILICONE-6, CTFA: POLYSILICONE-7 CTFA: POLYSILICONE-8 and CTFA: POLYSILICONE-13; cationic silicones e. g. CTFA: QUATERNIUM-80, cationic silicone polymers e. g. CTFA: POLYSILICONE-9, silicones, UV-filters, betaine, lanolin, lanolin derivatives, protein derivatives and protein hydrolysates, betaine, amino acids, cholesterol, pantothenic acid, vitamins, provitamins and plant extracts. The abbreviation “CTFA” refers to International Cosmetic Ingredient Dictionary and Handbook, Eighth Edition 2000 (ISBN 1-882621-22-0).

The anionic, nonionic, cationic and amphoteric or zwitterionic surface active agents are preferably selected from the groups consisting of:

a) anionic surface active agents, such as alkali, alkaline earth, ammonium or alkanolamine salts of alkyl sulfonates, alkyl sulfates and alkyl ether sulfates, such as sodium lauryl alcohol diglycol ether sulfate, sodium or triethanolamine salts of alkyl sulfates with 12 to 18 and preferably 12 to 14 carbon atoms, the sodium or triethanolamine salts of lauryl or tetradecyl ether sulfate, the disodium salt of the sulfosuccinic half ester of alkanolamides, soaps and polyether carboxylic acids;

b) nonionic surface active agents, such as ethoxylated fatty alcohols with 12 to 18 carbon atoms, such as lauryl, tetradecyl, cetyl and stearyl alcohol, ethoxylated with up to 40 moles of ethylene oxide per mole of fatty alcohol, alone or in a mixture, ethoxylated lanolin alcohols, ethoxylated lanolin, ethoxylated alkylphenols with 8 to 30 carbon atoms in the alkyl group and I to 1O ethylene oxide units in the molecule, fatty acid alkanolamides as well as ethoxylated sorbitol fatty acid esters;

c) cationic surface active agents, such as dilauryldimethylammonium chloride, chlorides or bromides of alkyldimethylbenzylammonium salts, alkyltrimethylammonium salts such acetyltrimethylammonium chloride or bromides, tetradecyltrimethylammonium chloride or bromides, alkyldimethylhydroxyelhylammonium chlorides or bromides, dialkyldimethylammonium chlorides or bromides, alkylpyridinium salts, such as lauryl- or cetylpyridinum chloride, alkylamidoethyltrimethylammonium ether sulfates, compounds with a cationic character, such as amine oxides, for example, alkyldimethylamine oxides or alkylaminoethyldimethylamine oxides and

d) amphoteric or zwitterionic surface active agents, such as carboxyl derivatives of imidazols, N-alkylbetaines, N-alkylamidobetaines, N-alkylsulfobetaines, N-alkylamino-propionates, alkyldimethylcarboxymethylammonium salts with 12 to 18 carbon atoms, as well as fatty acid alkylamidobetaines, such as fatty acid amidopropyldimethylamino acetic acid betaine.

The ready-for-use cosmetic composition is obtained by mixing at least 2 components, preferably up to 4 components, a short time (10 seconds to 20 minutes, preferably 5 to 20 minutes) before it is used on the hair or on the skin. It is most advantageous if the ready-for-use cosmetic composition is prepared by mixing two components immediately (5 minutes to 20 minutes) before use. These components can be present in the form of an aqueous composition, e. g. solution or emulsion as well as in thickened form on an aqueous basis, particularly as a cream, gel or paste. Advantageously, the cosmetic composition is manufactured as a multi-component package.

In the ready-for-use cosmetic composition, the cosmetic ingredients are used in amounts customary for this purpose. For example, wetting agents and emulsifiers are used in concentrations of from about 0.2 to about 30% by weight, alcohols in a concentration of from about 1 to about 80% by weight, hair conditioning or hair care components in a concentration of from about 0.1 to about 10% by weight, and thickening agents in concentrations of from about 0.1 to about 25% by weight.

In a preferred embodiment of the invention said cosmetic composition is a hair treatment composition. In the most preferred embodiment of the invention said hair treatment composition is a hair fixing composition for the purposes of permanent waving where the reduced hair fibers are oxidized with said hair fixing composition.

For enzymatic preparation of dehydroascorbic acid at the concentrations required to act as oxidizing agent in said cosmetic composition, oxygen must be added to the composition, which when this is done is preferred an aqueous solution. This may be done by shaking or stirring the composition in the presence of air, but it is most effectively accomplished by introducing a stream of oxygen-containing gas such as air into the composition either at 1 atmosphere or at elevated pressure. Another suitable method to get sufficient amounts of oxygen into the composition is the generation of oxygen-containing foam when the enzyme reaction is started. For this purpose, anionic, cationic, zwitterionic or nonionic surfactants are added to at least one of the components of the cosmetic composition, preferably hair fixative composition.

The oxygen may be present as pure oxygen gas, air, gas mixtures containing oxygen together with any non-reactive gas such as nitrogen, helium, argon, fluorohydrocarbons, and hydrocarbons (butane, propane, isobutane). Alternately, solids and liquids which decompose to give oxygen, such as mixtures of perfluorodecalin with oxygen and hydrogen peroxide with a catalyst for disproportionation may be employed as sources of oxygen. Oxygen can also first be dissolved in liquid media, including water, water-containing surfactants and other liquids. In particular, fluorinated hydrocarbons are known which can absorb high concentrations of dissolved oxygen. Such oxygen solutions may also contain such additives as fatty acids, higher molecular weight fatty alcohols, petroleum jelly, paraffin oil, polyethylene glycol, polyethylene glycol esters- or ethers, or alcohols such as ethanol, propanol and isopropanol, solubilizers, buffers, perfume oils, hair conditioning or hair care components, such as lanolin derivatives.

In a preferred embodiment Component 1 contains ascorbic acid, its derivatives or salts or the mixture thereof, preferably in an anhydrous form or with up to 10% by weight of water, as a powder, a granulate or tablet or micro-encapsulated or as a suspension. Component 1 is preferably used as a powder form. Component 1 may additionally contain at least one cosmetic ingredient.

Component 2 is a, preferably buffered, aqueous preparation which additionally contains at least one cosmetic ingredient.

Component 3, containing ascorbate oxidase can be an aqueous formulation, a powder, or else the enzyme can be immobilized on a solid support or on a soluble polymer.

Component 4. if present, is a source of oxygen or an oxygen equivalent.

Preferably at least one of the first 3 components contains a buffer for the pH adjustment of the hair fixative composition. The pH of the ready-for-use cosmetic composition—which may be used as a hair fixative—after mixing of the 3 components will be from about 1.5 to about 10, preferably from about 3.5 to about 8, most preferred from about 4.5 to about 6.5, by using an appropriate buffer.

These individual components may also be combined to give a fixative composition consisting of 1 or 2 multicomponent mixtures to be combined with an oxygen source immediately (10 seconds to 20 minutes) before use.

In another preferred embodiment of the invention, component 1 is an aqueous solution containing a buffer and component 2 contains ascorbic acid, its derivatives or salts and/or their mixture, preferably in an anhydrous form or with up to 10% by weight of water, as a powder, granules, tablet, microencapsulated or as a suspension. Beside this component 2 contains the ascorbate oxidase preferably also in an anhydrous form or with up to 10% by weight of water, as a powder, a granulate or tablet or microencapsulated or as a suspension. At least one of the two components contains at least one cosmetic ingredient.

In a most preferred embodiment of the invention the cosmetic composition is prepared by mixing two components wherein component 1 is a mixture of all substances in dry solid form which are ascorbic acid, buffer salts, enzyme and cosmetic additives and component 2 is an aqueous or aqueous-alcoholic composition, preferably an aqueous solution. Component 1 and component 2 are mixed immediately before leaving said mixture come into contact with oxygen.

The following paragraphs will further describe the reaction components, referring to the version of the hair fixative formulation consisting of 4 separate components as mentioned before. For convenience, the components so described can be combined as component mixtures prior to use as described in the preceeding paragraph.

Each of the components of the hair fixative composition may contain all additives, customary for cosmetic ingredients.

The liquid aqueous components of the hair fixative composition can be present in the form of an aqueous solution or of an aqueous emulsion as well as in thickened form on an aqueous basis, particularly as a cream, gel or paste. Alternately, chemicals, buffer salts, enzymes, stabilizers and cosmetic modifiers may initially be in solid forms such as powders, granules, resins and coatings on other solids; to be mixed with water or other aqueous mixtures for the oxidation of ascorbic acid.

In this embodiment component 1 of the fixative formulation contains ascorbic acid or its derivatives or its salts or a mixture thereof in an appropriate concentration so that the ready to use fixative formulation (immediately after mixing of all components) preferably contains from about 0.1 to about 20% by weight, preferably from about 0.5 to about 10% by weight and especially from about 1 to about 4% by weight of ascorbic acid or its derivatives or its salts or a mixture thereof.

Additives which stabilize ascorbate, such as nonvolatile thiols, and deferoxamine mesylate, may be included in the component containing the ascorbic acid or its derivative or its salt or a mixture-thereof as well as liquid media such as vegetable oils. Solid ascorbic acid or ascorbate salts may be surface-treated or encapsulated to prevent oxidation during storage and to keep out of contact from ascorbate oxidase which might be stored in a solid mixture together with ascorbic acid in a cosmetic product. Preferably the solid ascorbic acid is coated with a material such as appropriate polymers, surfactants waxes and/or emulsifiers and fats.

Compounds required to adjust the final pH of the ascorbic acid oxidation mixture are preferably included in component 2.

The pH of the fixative formulation after mixing of the all components ranges form about 1.5 to about 10, preferably from about 3.5 to about 8 and most preferred form about 4.5 to about 6.5. The pH is adjusted with conventional bases, acids and buffering materials, such as ammonia, alkali hydroxides, alkali carbonates, alkali hydrogen carbonates, citrate buffer, phosphoric acid and its salts, citrate acid and its salts.

Component 3 contains the enzyme required for oxidation, preferably ascorbate oxidase.

Component 4 contains the oxygen or oxygen equivalent required for converting ascorbic acid to dehydroascorbic acid.

Component 4 of the fixative composition may additionally contain conventional oxidizing agents, such as hydrogen peroxide, peroxide salts or bromates.

Each of the components of the fixative composition may contain ingredients, which are customary in cosmetic preparations for the hair as listed before. These additives are preferably contained in the aqueous component 2 of the fixative composition, either in solution or as an aqueous emulsion.

These wetting agents and emulsifiers are preferably contained in the aqueous component 2 of the fixative formulation.

Solid or liquid additives, compatible with ascorbic acid and with ascorbate oxidase, may be contained together with the ascorbic acid in component 1 or with ascorbate oxidase component 3, while the remaining additives are preferably contained in the liquid component 2.

The application temperature of the fixative composition ranges from about 10 degree C to about 60 degree C. and preferably from about 20 degree C. to about 55 degree C. and especially from 30 degree C. to 50 degree C. The duration of action ranges from about 1 to about 45 minutes, preferably from about 3 to about 25 minutes and especially from 5 to 15 minutes.

The ascorbic acid, its derivatives or its salt or its mixture is used in component 1 alone or as a mixture with the additives, conventionally used in cosmetics, in an anhydrous medium, preferably as a dust-free powder, granulate or as a tablet.

The ascorbate oxidase is used in component 3 alone or as a mixture with the ingredients, conventionally used in cosmetics, in an anhydrous medium, preferably as a dust-free powder, granulate or as a tablet.

When the ready-to-use fixative composition is prepared by mixing component 1 and component 3 with component 2, the resulting mixture contains 0.1 to 20% by weight, preferably 0.5 to 10% by weight, and most preferred 1 to 4% by weight of ascorbic acid, its derivative and/or its salt. The mixture than contains I to 2000 ppm of ascorbate oxidase protein. In preferred embodiment the ready-to-use fixative composition contains 2 to 500 ppm of the enzyme protein.

A further object of the present invention is a method for preparing a ready-to-use cosmetic composition for the oxidative treatment of skin or hair, said method comprising the steps of:
(i) providing a component (A) comprising at least one compound selected from the group of ascorbic acid, ascorbic acid derivative and ascorbic acid salt as well as at least one cosmetic ingredient;
(ii) providing a component (B) comprising an enzyme that catalyzes the enzymatical oxidation of said of ascorbic acid, ascorbic acid derivative and ascorbic acid salt;
(iii) providing a component (C) comprising oxygen;
(iv) mixing components (A) and (B) from about 1 minute to about 20 minutes before application,
(v) mixing component (C) intensely with the mixture of components (A) and (B).

In a preferred embodiment of the invention is a method for preparing a ready-to-use cosmetic composition for the oxidative treatment of skin or hair, said method comprising the steps of:
(i) providing a component (A′), comprising in dry solid form:
at least one compound selected from the group of ascorbic acid, ascorbic acid derivative and ascorbic acid salt;
at least an enzyme that catalyzes the enzymatical oxidation of said of ascorbic acid, ascorbic acid derivative and ascorbic acid salt; and
at least one cosmetic ingredient;
(ii) providing a component (B′), comprising an aqueous or aqueous-alcoholic composition;
(iii) providing a component (C′) comprising oxygen;
(iv) mixing components (A′) and (B′) from about 1 minute to about 20 minutes before application,
(v) leaving component (C′) to come into contact intensely with the mixture of components (A) and (B).

In a preferred embodiment of the inventive method for preparing a ready-to-use cosmetic composition the oxygen is present in the form of air, purified oxygen gas, an oxygen gas-containing mixture or any other oxygen gas releasing compound.

It is further preferred that said step (v) is carried out in a pressurized container.

In the most preferred embodiment step (v) is carried out in presence of a solution of one or more anionic, cationic, zwitterionic or nonionic surfactants appropriate to provide an oxygenated foam.

It is further preferred that the oxygen in step (v) is chemically or physically bound in an oxygen containing compound.

A further object of the present invention is a method for the oxidative treatment of keratin, said method comprising the steps of:
(a) providing a cosmetic composition as disclosed above,
(b) applying said cosmetic composition to the keratin,
(c) allowing said cosmetic composition to act on the keratin for a sufficient time, and
(d) rinsing the keratin.

In a preferred embodiment of said method for the oxidative treatment of keratin said keratin is hair.

A further object of the present invention is a method for permanent hair shaping, for which the hair, before and/or after it is brought into the desired shape, is treated with a keratin-reducing, permanent shaping agent for a period of time which is sufficient to shape the hair, rinsed, then treated oxidatively with a fixative, rinsed, subsequently styled and then dried, wherein the fixative, described above, is used for the oxidative treatment. Preferably the rinsing is carried out with water.

In a preferred embodiment of said method for permanently shaping hair, said method comprising the steps of:
a) bringing the hair into a desired shape;
b) applying a keratin-reducing composition to the hair and allowing the keratin-reducing composition to act on the hair for a period of action sufficient for the permanent shaping of hair;
c) rinsing the hair after the applying and allowing of step b);
d) providing an oxidative hair fixing composition as defined in claim 22,
e) after the rinsing of step c), applying said oxidative hair fixing composition to the hair and allowing said oxidative hair fixing composition to act on the hair for a time sufficient for fixing of the hair in the desired shape; and
f) after the applying and the allowing of step e), rinsing the hair again.

In a special embodiment of the inventive method, the hair is first treated with the keratin-reducing permanent waving agent for a period of time which is sufficient to shape the hair, the permanent waving agent is rinsed out thereafter, subsequently the hair is treated with the fixative, which is described above and based on enzymatically-generated dehydroascorbic acid, its derivatives and/or salts as oxidizing agent (pre-fixed) and then treated with a fixative based on hydrogen peroxides or bromate (post-fixation). It is particularly advantageous if the fixative, for the post-fixation, has a lower concentration of oxidizing agent than is customary for such fixatives; for example, the concentration of hydrogen peroxide is only 0.1 to 1% by weight and of the bromate only 1 to 5% by weight.

In a preferred embodiment said method for permanently waving hair comprising the steps of:
a) bringing the hair into a desired shape;
b) applying a keratin-reducing composition to the hair and allowing the keratin-reducing composition to act on the hair for a period of action sufficient for the permanent waving;
c) rinsing the hair after step b);
d) providing an oxidative hair fixing composition as defined in claim 22,
e) after the rinsing of step c), applying said oxidative composition as a pre-fixing composition to the hair and allowing said oxidative pre-fixing composition to act on the hair for a time sufficient for pre-fixing the hair; and
f) after the pre-fixing of the hair of step e), treating of the hair with an oxidative post-fixing composition for post-fixing the hair, said oxidative post-fixing composition containing from 0.1 to 1 percent by weight of hydrogen peroxide or from I to 5 percent by weight of bromate as oxidizing agent.

For the method according to the invention, the hair is washed, massaged with a towel, optionally pre-moistened with a portion of the keratin-reducing permanent waving agent, divided into individual strands and wound on curlers. Depending on whether permanent waving is desired or the hair is to be straightened, the diameter of the curler is either about 5 to 13 mm or about 15 to 35 mm. An amount of agent, adequate for permanent waving, is subsequently applied on the hair in curlers. The total amount of agent, required for the permanent waving, generally is from about 80 g to about 100 g.

The permanent waving agents, which can be used for the inventive method, usually contain keratin-reducing compounds, such as certain thiol compounds, particularly thioglycolic acid, thioglycerin, cysteine, cysteamine as well as salts or esters of mercapto carboxylic acids. These permanent waving agents contain the keratin-reducing compounds in amounts, customary for such agents. For example, the ammonium salts of thioglycolic acid or thiolactic acid are contained in an amount of about 2 to 12 percent by weight. The pH of these permanent waving agents generally is about 7 to 11. The pH preferably is adjusted with ammonia, monoethanolamine, ammonium carbonate or ammonium hydrogen carbonate. When adjusting the permanent waving agent to an acidic pH of, for example, 6.5 to 6.9, preferably esters of mercaptocarboxylic acids, such as monothioglycolic acid glycol esters or glycerin esters are used in a concentration of from about 2 to about 25% by weight.

The permanent waving agents furthermore may contain all additives, customary for such agents, such as swelling materials, penetration materials, thickening agents, wetting agents and emulsifiers, alcohols, solubilizers, stabilizers, dyes, perfume oils as well as hair-conditioning or hair care components. The additives, named above, are used in amounts customary for such purposes. For example, the wetting agents and emulsifiers are used in concentrations of about 0.2 to 30% by weight, while the thickeners may be contained in an amount of about 0.1 to 25% by weight in the permanent waving agent.

The reducing agent, used in the inventive method, may be present in the form of an aqueous solution or emulsion, as well as in thickened form on an aqueous basis, especially as a cream, gel or paste or in the form of an aerosol foam.

After a period of action, which is sufficient for permanent waving and depends on the nature of the hair, the pH and the effectiveness of the reducing agent as well as on the application temperature, and amounts to 5 to 45 minutes (5 to 30 minutes with heat; 20 to 45 minutes without heat), the hair is rinsed with water and then treated oxidatively with 50 g to 350 g and preferably with 80 g to 200 g of the ready-to-use fixative formulation described above.

After the fixative composition has been allowed to act for 1 to 45 minutes, preferably 3 to 25 minutes and especially 5 to 15 minutes, the curlers are removed and the unrolled hair, if necessary, is treated once again oxidatively with the fixative. The hair is then rinsed preferably with water, styled and dried.

The shape of hair, so treated, is uniformly altered, and the new shape becomes increasingly durable after repeated permanent wave treatments. In contrast to dehydroascorbic acid-fixed hair, hair which was fixed with peroxide and has a clearly detectable shift in coloring the direction of red and yellow the values for the fixative, use pursuant to the invention, lie within the range of untreated hair strands. In addition, the cysteic acid content of the dehydroascorbic acid fixed hair, is clearly less than that of hair, which was treated with a fixative based on hydrogen peroxide and bromate. In addition, the oxidation with the fixative, described here, does not result in an unpleasant mercaptan odor.

The outstanding results concerning reduced hair damage, achieved with the enzymatically generated dehydroascorbic acid as the inventive agent described herein are comparable with those achieved with the dehydroascorbic acid as described in a former patent application U.S. Pat. No. 6,506,373.

In the case of peroxide or bromate oxidative hair damage occured whereas the use dehydroascorbic acid does not cause oxidative damage. The bundle tensile strength of the strands did not decrease with increasing number of waving treatments when dehydroascorbic acid was used as an oxidizing agent, which shows that no increase in hair damage was observed even when used repeatedly. In the case of repeated use of bromate or peroxide the bundle tensile strength of the strands decreased which is an evidence for increased hair damage caused by bromate or peroxide.

### EXAMPLES

### Example 1

Comparison of the Wave Strength

Wave strength was evaluated visually with permanent waved hair strands which were fixed with enzymatically generated dehydroascorbic acid immediately produced before application on reduced hair. The resulted visibly determined wave strands were compared with those determined with hair strands fixed with purchased dehydroascorbic acid (as a control) and other hair strands fixed with a buffer. For this purpose 16.5 centimeters of long, prebleached and thus damaged strands of hair of Central European origin, were rolled in wet condition onto standard spiral curlers and after conditioning in a climate controlled room (temperature: 20° C.; air humidity: 65%) were treated with a solution containing 9.5 wt.%. ammonium thioglycolate set to pH 8. The quantity of wave solution applied was calculated at a ratio 1:1.2 (1 g hair: 1.2 ml waving solution). This ratio corresponds to 50 ml of permanent waving solution per head with an average weight of about 30 g of hair per head. The reaction time was set for 20 min; the reaction temperature was 45° C.

During that time the dehydroascorbic acid was enzymatically generated by mixing the following components in an appropriate way as described below:

Component 1

| | | |
|---|---|---|
| 2.50 | g | ascorbic acid, anhydrous powder |

Component 2

| | | |
|---|---|---|
| 0.50 | g | PEG-40 Hydrogenated Castor Oil |
| 0.20 | g | perfume oil |
| | | phosphate buffer to a pH of 5.5 |
| 96.50 | g | water |

Component 3

| | | |
|---|---|---|
| 0.05 | g | ascorbate oxidase, lyophilized |
| 0.20 | g | Polyethylenglykol 2000 |

Component 4
4 to 6 liters of air at normal pressure

Component 1 consisting of 2.5 g of ascorbic acid (anhydrous powder) were diluted in component 2 (aqueous solution adjusted to pH 6 containing additional cosmetic ingredients as specified above). Then Component 3 (containing 50 mg of lyophilized ascorbate oxidase as specified above) was added and a foam was made by bubbling approximately 6 liters of air through the solution over a time period of 10 min. After 10 min the concentration of dehydroascorbic acid in the ready-to-use fixative composition was 1.5% by weight. Then the reduced hair strands were treated with this composition.

Subsequently further reduced hair strands were treated with a phosphate buffer solution (0.1 mol/liter phosphate buffer, pH 5.5) and an aqueous buffered solution of 2,5% purchased dehydroascorbic acid for control measurements (contains additionally 0.1 mol/liter phosphate buffer, pH 5.5). The reduced hair strands were wrapped around 1 cm diameter curlers and were then immersed in the fixative solutions over 3 minutes and were stored for further 7 min out of solution with the fixative formulation on the hair. Further on the curlers were rinsed with water and dried. Then the curlers were removed and the hair strands were suspended for four hours in a water bath (water bath temperature: 40° C.). Then the hair strands were hang out in a climate controlled room (temperature: 20° C.; air humidity: 65%) and after 12 h the resulting curls were visually evaluated. It was stated that both the hair strands treated with enzymatically produced dehydroascorbic acid according to the invention and the hair strands treated with purchased dehydroascorbic acid are well transformed to curls and the lengths of these curls were equal showing a similar efficacy of prior-to-application enzymatically produced dehydroascorbic acid and the purchased dehydroascorbic acid.

The curl transformation of the hair strands previously treated with buffer were much weaker. These hair strands were much extended in length in comparison to those previously treated with the dehydroascorbic acid containing formulations. From this experimental result it was concluded that dehydroascorbic acid can fixate the hair by working as an oxidant whereas pure buffer did not fixate because there was not added any oxidants.

This is an additional evidence for the fixating efficacy of dehydroascorbic acid which perfectly comply with results previously generated by extensive investigations (U.S. Pat. No. 6,505,373). Particularly it shows that enzymatically prior-to-application produced dehydroascorbic acid can fixate reduced hair in the same excellence as we know it from purchased dehydroascorbic acid (U.S. Pat. No. 6,505,373).

### Example 2

Hair samples are first prepared and reduced as described in example 1. The reaction to produce the fixative agent dehydroascorbic acid enzymatically is performed by mixing the components 1 to 3 specified in this example in the manner described in example 1, where it is mixed in a pressure resistant can. Compressed air with a pressure of 10 bar at 25° C. was brought into contact with the liquid solution over 10 min by acting with roughly constant pressure.

Component 1

| | | |
|---|---|---|
| 2.50 | g | ascorbic acid, anhydrous powder |

Component 2

| | | |
|---|---|---|
| 0.50 | g | PEG-25 Stearate |
| 0.20 | g | perfume oil |
| | | phosphate buffer to a pH of 4.5 |
| 96.50 | g | water |

Component 3

| | | |
|---|---|---|
| 0.02 | g | ascorbate oxidase |
| 0.30 | g | Polyethylenglykol 350 |

Component 4
250 ml of compressed air at 8 bar

The pH of the ready-for-use fixative is adjusted to a value of 4.5 with a phosphate buffer. Then the reduced hair strands were rinsed with water and treated with the formulation containing high amounts of enzymatically produced dehydroascorbic acid. Therefore the reduced hair strands curled on curlers were immersed in the fixative solutions over 3 minutes and were left on the hair for a further 7 min out of solution, after which the curlers were rinsed with water and dried.

Then the hair strands were hang out in a climate controlled room (temperature: 20° C.; air humidity: 65%) and after 12 h the resulted curls were visually evaluated.

The hair, so treated, showed a good transformation and a durability of curls over months, exhibits a good general state, is not bleached, is free of any disturbing mercaptan odor and the curls are stable over months.

### Example 3

Hair samples are first prepared and reduced as described in example 1. The reaction to produce the fixative composition dehydroascorbic acid enzymatically is performed by mixing the components 1 and 2 specified in this example in a pressure resistant can equipped with a device to generate a foam. The pH of the ready-for-use fixative is adjusted to a value of 4.5 with a phosphate buffer. Then a foam is produced with compressed air (pressure 10 bar at 25° C.) using an appropriate outlet. After 10 min of reaction time the resulting formulation contains high amounts of enzymatically produced dehydroascorbic acid.

Component 1

| | | |
|---|---|---|
| 0.20 | g | PEG-40 Stearate |
| 0.10 | g | Sodium laureth sulfate |
| 0.20 | g | perfume oil |
| | | phosphate buffer to a pH of 4.5 |
| 96.00 | g | water |

Component 2

| | | |
|---|---|---|
| 0.02 | g | ascorbate oxidase, lyophilized powder |
| 5.00 | g | Ceteareth-12 coated ascorbic acid, dry powder |

Component 3
air at pressure of 10 bar at 25° C.

For fixation the reduced hair strands which were curled on curlers were immersed in the fixative solutions over 3 minutes and were stored for further 7 min out of solution with the fixative formulation on the hair. Subsequently, the hair samples were rinsed with water and dried on the curlers.

The dried hair strands were hung out in a climate controlled room (temperature: 20° C.; air humidity: 65%) and after 12 h the resulted curls were visually evaluated.

The hair, so treated, showed a good transformation and a durability over months, is not bleached and is free of disturbing mercaptan odor.

### Example 4

Hair samples are first prepared and reduced as described in example 1. The reaction to produce the fixative agent dehydroascorbic acid enzymatically is performed by mixing the components 1 to 4 specified in this example in the manner described in example 1. The pH of the ready-for-use fixative is adjusted to a value of 4.5 with a phosphate buffer. After 12 min of reaction time appropriate amounts of dehydroascorbic acid were generated and the fixative formulation can be applied on reduced hair.

Component 1

| | | |
|---|---|---|
| 2.50 | g | ascorbic acid, anhydrous powder |

Component 2

| | | |
|---|---|---|
| 0.50 | g | PEG-25 Stearate |
| 0.20 | g | perfume oil |
| | | phosphate buffer to a pH of 4.5 |
| 96.50 | g | water |

Component 3

| | | |
|---|---|---|
| 0.02 | g | ascorbate oxidase, lyophilized powder |
| 0.30 | g | Polyethylenglykol 350 |

Component 4

| | | |
|---|---|---|
| 1.5 | g | Fiflow PB 140 (Creatin Couleurs Company) consisting |
| | | of 62.5 wt. % Perfluorodecalin and 37.5 wt. % Oxygen |

The reduced hair strands were rinsed with water and then treated with the formulation containing high amounts of enzymatically produced dehydroascorbic acid. Therefore the reduced hair strands curled on curlers were immersed in the fixative solutions over 3 minutes and were stored for further 7 min out of solution with the fixative formulation on the hair. Further on the curlers were rinsed with water and dried. Then the hair strands were hang out in a climate controlled room (temperature: 20° C.; air humidity: 65%) and after 12 h the resulted curls were visually evaluated.

The hair, so treated, showed a good transformation and a durability of curls over months, exhibits a good general state, is not bleached and is free of any disturbing mercaptan odor.

### Example 5

Hair samples are first prepared and reduced as described in example 1. The reaction to produce the fixative agent dehydroascorbic acid enzymatically is performed by mixing the components I and 2 specified in this example and by bubbling 6 liters of air (component 3) through the solution over a time period of 10 min. The pH of the ready-for-use fixative is adjusted to a value of 4.5 with a phosphate buffer. After 10 min of reaction time appropriate amounts of dehydroascorbic acid were generated and the fixative formulation can be applied on reduced hair.

Component 1

| | | |
|---|---|---|
| 0.20 | g | PEG-40 Stearate |
| 0.10 | g | Sodium laureth sulfate |
| 0.20 | g | perfume oil |
| | | phosphate buffer to a pH of 4.5 |
| 96.00 | g | water |

Component 2

| | | |
|---|---|---|
| 0.020 | g | PEG 5000 coated lyophilized ascorbate oxidase |
| 5.000 | g | Ascorbic acid, dry powder |

Component 3
6 liters of air at normal pressure

For fixation the reduced hair strands which were curled on curlers were immersed in the fixative solutions over 3 minutes and were stored for further 7 min out of the solution with the fixative formulation on the hair. Subsequently, the hair samples were rinsed with water and dried on the curlers. The dried hair strands were hung out in a climate controlled room (temperature: 20° C.; air humidity: 65%) and after 12 h the resulted curls were visually evaluated.

The hair, so treated, showed a good transformation and a stability of curls over months, is not bleached and is free of disturbing mercaptan odor.

### Example 6

A solution is prepared from 0.620 g of ascorbic acid, 0.435 g of K₂HPO₄ and water to give 25 ml of a buffered 2.5% by weight ascorbic solution with pH 4.55. At a temperature of 27° C., solution of zucchini ascorbate oxidase is added to give (˜20 ppm enzyme protein, and a rapid stream of oxygen gas is introduced through two polyethylene fritted filters. Aliquots are removed and diluted at intervals over the next 30 minutes and are assayed for ascorbic acid following further dilution with 0.2 M HCl (ε₂₄₅=10⁴ M⁻¹) and for dehydroascorbic acid. After 10 minutes, the concentration of dehydroascorbic acid was about 1% by weight. After 30 minutes, the pH of the solution is 6.3, and the oxygen stream is shut off.

Three samples of washed, reduced hair, previously prepared as in Example 1, are treated with the solution at 45° C. for 15 minutes, then the fixed hair sample is thoroughly rinsed with water and dried. An identical enzyme reaction mixture-is treated with oxygen for only 10 minutes before it is used to fix three more reduced hair samples at 45° C. (15 minutes). The fixed, rinsed and dried hair samples are suspended in a warm water bath (40° C.) with standardized weights (100 mg) attached to the bottom of each set of hair strands. Changes in shape and length of curled hair are monitored by using a video camera over the course of four hours.

Permanent wave standards are prepared in parallel using commercial 2.5% by weight bis-dehydroascorbic acid and a standard peroxide formula as fixatives, also at 45° C. Curl stability for each sample is judged by the degree of extension according to standard methodology (see U.S. Pat. No. 6,153,180). After 4 hours, average curl stability values for the hair samples treated with commercial available dehydroascorbic acid and with enzymatically generated dehydroascorbic acid were the same within experimental error.

### Example 7

Two enzyme reaction mixtures were prepared as in Example 6, then 0.1% lauryl ether sulfate (Texapon N25) and 0.5% polyethylene glycol-modified castor oil (Cremophor RH40) were added to one mixture each. After 20 minutes, each solution was used to fix reduced hair samples and the curl stability was determined as before.

### Example 8

Three enzyme reaction solutions containing 0.5% Cremophor RH40, prepared as in Example 6, are placed in 6 cm diameter glass pressure vessels (Ace Glass Co. Cat # 8648-140) with 2 cm magnetic stirring bars, then oxygen is introduced at a pressure of 4 bar and the mixtures are vigorously stirred. The pressure is released and the dehydroascorbic acid concentrations are determined at the times specified

After 4 hours, average curl stability values for the hair samples treated with commercial available dehydroascorbic acid and with enzymatically generated dehydroascorbic acid according to example 8 were the same within experimental error.

1. Cosmetic composition comprising
(a) at least one compound selected from the group consisting of ascorbic acid, ascorbic acid derivative and an ascorbic acid salt,
(b) an enzyme that catalyzes the enzymatical oxidation of said of ascorbic acid, ascorbic acid derivative or ascorbic acid salt and
(c) at least one cosmetic ingredient.

2. The composition as defined in claim 1 wherein the pH is from 1.5 to 10.

3. The-composition as defined in claim 1 wherein the pH is from 3.5 to 8.

4. The composition as defined in claim 1, wherein the composition is a hair treatment composition.

5. The composition as defined in claim 1, in which said enzyme is selected from an oxygen-utilizing ascorbate oxidase.

6. The composition as defined in claim 5, wherein the enzyme belongs to the Enzyme Commission class [1.10.3.3].

7. The composition as defined in claim 5, wherein the enzyme is of plant origin.

8. The composition as defined in claim 7, wherein the enzyme is derived from Arabidopsis, Brassica, Cucumis, Cucurbita, Myrothecium, Nicotiana, Oryza, Sinapis, Titicum species.

9. The composition as defined in claim 8, wherein the enzyme is derived from Cucurbita pepo medullosa (zucchini).

10. The composition as defined in claim 1, wherein the enzyme is a cloned enzyme expressed in cultured cells or an organism other than that from which the gene for the enzyme is derived.

11. The composition as defined in claim 10, wherein the enzyme is expressed in bacterial or yeast cultures.

12. The composition as defined in claim 10, wherein the enzyme is derived from bacterial or fungal species.

13. The composition as defined in claim 5, wherein the enzyme is stabilized by a substance selected from the group consisting of buffers, glycerol, polyhydroxy compounds, metal chelating agents, thiols, polyethylene glycol and nonreactive proteins.

14. The composition as defined in claim 5, wherein the enzyme is stabilized by immobilisation.

15. The composition-as defined in claim 14, wherein the immobilisation is made by covalently attaching the enzymes to a solid support selected from the group consisting of microparticles of surface-modified silica, alumina, glass, oxirane-modified polymethacrylate, carboxyalkylcellulose, aminoalkylsilica, aminoalkyl glass, aminoalkyl cellulose, carboxyalkyl cellulose, dialkylamino-substituted cellulose, polyethylene glycol (PEG), polyacrylic acid, polyvinyl alcohol, polyethyleneimine, dextran, gelatin and uricase.

16. The composition as defined in claim 1, wherein said enzyme is contained in a concentration of from 1 to 10000 ppm, whereas this is the concentration of ascorbate oxidase in the ready-to-use composition immediately after mixing of all components of this composition.

17. The composition as defined in claim 1, wherein said enzyme is contained in a concentration of from 10 to 1000 ppm, whereas this is the concentration of ascorbate oxidase in the ready-to-use composition immediately after mixing of all components of this composition.

18. The composition defined in claim 1, wherein the ascorbic acid, ascorbic acid derivative and/or ascorbic acid salt is present as an anhydrous powder, a granulate, a coated material, a tablet or micro-encapsulated.

19. The composition as defined in claim 1, wherein the cosmetic ingredient is selected from the group consisting of thickening agents, such as bentonite, kaolin, fatty acids, starch, guar gum, high molecular weight fatty alcohols, polyacrylic acid and its derivatives, cellulose derivatives, alginates, Vaseline, paraffin oils, wetting agents or emulsifiers from the classes of anionic, cationic, amphoteric or nonionic surface-active substances, such as fatty alcohol sulfates, fatty alcohol ether sulfates, alkylsulfonates, alkylbenzenesulfates, quaternary ammonium salts, alkylbetaines, ethoxylated alkylphenols, fatty acid alkanolamides or ethoxylated fatty esters, furthermore opacifiers, such as polyethylene glycol esters, alcohols, such as ethanol, propanol, isopropanol, polyols, such as ethylene glycol: 1,2- or 1,3-dihydroxy-propane, 1,2-, 1,3- or 1,4-dihydroxy-butane, 1,2-, 1,3-, 1,4- or 1,5-dihydroxy-pentane and glycerin, sugars, such as D-glucose, solubilizers, stabilizers, buffering substances, perfume oils, dyes as well as hair conditioning and hair care components, such as cationic polymers, silicone polymers, cationic silicone polymers, UV-filters, betaine, lanolin, lanolin derivatives, protein derivatives and protein hydrolysates, amino acids, cholesterol, pantothenic acid, vitamins, provitamins and plant-extracts.

20. The composition as defined in claim 1, wherein the ascorbic acid, ascorbic acid derivative and/or ascorbic acid salt is present in a concentration of from about 0.1 to about 20% by weight in the ready-for-use composition.

21. The composition/as defined in claim 20, wherein the ascorbic acid, ascorbic acid derivative and/or ascorbic acid salt is present in a concentration of from about 0.5 to about 10% by weight in the ready-for-use composition.

22. The composition as defined in claim 1, Of whereby said cosmetic composition is a hair fixing composition for permanently shaping hair.

23. The composition as defined in claim 1, whereby said composition is an aqueous solution.

24. A method for preparing a ready-to-use cosmetic composition for the oxidative treatment of skin or hair, said method comprising the steps of:
(i) providing a component (A) comprising at least one compound selected from the group of ascorbic acid, ascorbic acid derivative and ascorbic acid salt as well as at least one cosmetic ingredient;
(ii) providing a component (B) comprising an enzyme that catalyzes the enzymatical oxidation of said of ascorbic acid, ascorbic acid derivative and ascorbitc acid salt;
(iii) providing a component (C) comprising oxygen;
(iv) mixing components (A) and (B) from about 1 minute to about 20 minutes before application,
(v) mixing component (C) intensely with the mixture of components (A) and (B).

25. A method for preparing a ready-to-use cosmetic composition for the oxidative treatment of skin or hair, said method comprising the steps of:
(i) providing a component (A′), comprising in dry solid form: at least one compound selected from the group of ascorbic acid, ascorbic acid derivative and ascorbic acid salt;
at least an enzyme that catalyzes the enzymatical oxidation of said of ascorbic acid, ascorbic acid derivative and ascorbic acid salt; and
at least one cosmetic ingredient;
(ii) providing a component (B′), comprising an aqueous or aqueous-alcoholic composition;
(iii) providing a component (C′) comprising oxygen;
(iv) mixing components (A′) and (B′) from about 1 minute to about 20 minutes before application,
(v) leaving component (C′) to come into contact intensely with the mixture of components (A) and (B).

26. The method as defined in claim 24 or 25 wherein the oxygen is present in the form of air, purified oxygen gas, an oxygen containing mixture or any other oxygen gas releasing compound.

27. The method as defined in claim 24 or 25 wherein step (v) is carried out in a pressurized container.

28. The method as defined in claim 24 or 25 wherein step (v) is carried out in presence of a solution of one or more anionic, cationic, zwitterionic or nonionic surfactants appropriate to provide an oxygenated foam.

29. The method as defied in claim 24 or 25 wherein the oxygen in step (v) is chemically or physically bound in an oxygen containing compound.

30. A method for the oxidative treatment of keratin, said method comprising the steps of:
a) providing said cosmetic composition of
(b) applying said cosmetic composition to the keratin,
(c) allowing said cosmetic composition to act on the keratin for a sufficient time, and
(d) rinsing the keratin.

31. A method as defined in claim 30, whereby the keratin is hair.

32. A method as defined in claim 30 whereby the oxidative treatment is a oxidative post treatment of reduced hair in the process of permanent shaping of hair.

33. A method as defined in claim 30, whereby the sufficient time in step (c) is from about 5 minutes to about 25 minutes.

34. A method for permanently shaping hair, said method comprising the steps of:
a) bringing the hair into a desired shape;
b) applying a keratin-reducing composition to the hair and allowing the keratin-reducing composition to act on the hair for a period of action sufficient for the permanent shaping of hair;
c) rinsing the hair after the applying and allowing of step b);
d) providing an oxidative hair fixing composition as defined in claim 22,
e) after the rinsing of step c), applying said oxidative hair fixing composition to the hair and allowing said oxidative hair fixing composition to act on the hair for a time sufficient for fixing of the hair in the desired shape; and
f) after the applying and the allowing of step e), rinsing the hair again.

35. A method for permanently waving hair, said method comprising the steps of:
a) bringing the hair into a desired shape;
b) applying a keratin-reducing composition to the hair and allowing the keratin-reducing composition to act on the hair for a period of action sufficient for the permanent waving;
c) rinsing the hair after step b);
d) providing an oxidative hair fixing composition as defined in claim 22,
e) after the rinsing of step c), applying said oxidative composition as a pre-fixing composition to the hair and allowing said oxidative pre-fixing composition to act on the hair for a time sufficient for pre-fixing the hair; and
f) after the pre-fixing of the hair of step e), treating of the hair with an oxidative post-fixing composition for post-fixing the hair, said oxidative post-fixing composition containing from 0.1 to 1 percent by weight of hydrogen peroxide or from 1 to 5 percent by weight of bromate as oxidizing agent.

36. The method as defined in claim 35, wherein said oxidative pre-fixing composition is a solution and has a pH of 3.5 to 9.
